(19) United States
(12) Patent Application Publication
(10) Pub. No.: US 2025/0325608 A1
(43) Pub. Date: Oct. 23, 2025

(54) CANCER THERAPY

(71) Applicant: The Regents of the University of California, Oakland, CA (US)

(72) Inventors: Nicole F. Steinmetz, San Diego, CA (US); Edward Koellhoffer, San Diego, CA (US)

(21) Appl. No.: 18/855,896

(22) PCT Filed: Apr. 12, 2023
(86) PCT No.: PCT/US2023/018332

§ 371 Date: Oct. 10, 2024

Related U.S. Application Data
(60) Provisional application No. 63/330,741, filed on Apr. 13, 2022.

(51) Int. Cl. A61K 35/768; A61K 39/00; A61K 39/12; A61K 39/395; A61P 35/00

(52) U.S. Cl. CPC A61K 35/768; A61K 39/12; A61K 39/39558; A61P 35/00; A61K 2039/585

(57) ABSTRACT

This disclosure provides compositions and combinations for treating cancer. The compositions or combinations comprise a plant virus and a natural killer (NK) cell agonist.

### CROSS-REFERENCE TO RELATED APPLICATION

This application claims priority under 35 U.S.C. § 119 (e) to U.S. Provisional Application No. 63/330,741, filed Apr. 13, 2022, the contents of which are incorporated herein by reference in their entireties.

### STATEMENT OF GOVERNMENT SUPPORT

This invention was made with government support under CA253615, CA218292, EB005970, and CA224605 awarded by the National Institutes of Health. The government has certain rights in the invention.

### BACKGROUND

Cancer remains one of the leading causes of death in the United States, and can inflict devastating morbidity and costly challenges on patients and their families. The need to continue to develop cancer immunotherapies and cancer vaccines remains persistent. Cancer immunotherapies are a unique approach that are aimed to utilize the immune system in recognizing and eliminating transformed cancer cells^{1,2}. However, malignant tumors themselves are poised to evade a robust immune response through a variety of mechanisms. Within the tumor microenvironment, cancers secrete a number of immunosuppressive cytokines and signaling molecules to suppress the innate immune cells and ultimately evade immunological response³⁻⁵.

Natural Killer or NK cells have ability to kill tumor cells-however, exploiting NK cells in cancer immunotherapy is met by two major challenges: (1) recruiting NK cells to the tumor microenvironment, and (2) stimulating NK cells to overcome the immunosuppressive hurdle of the tumor microenvironment to enact NK cell function. This discloses addresses these technical challenges in the art and provides related advantages as well.

### SUMMARY OF THE DISCLOSURE

Provided herein is a composition or combination comprising, or consisting essentially of, or yet further consisting of a natural killer (NK) cell agonist and a plant virus selected from the group of Cowpea chlorotic mottle virus (CCMV), Cowpea mosaic virus (CPMV), Physalis mottle virus (PhMV) and Sesbania mosaic virus (SeMV). In one aspect, the NK cell agonist comprises an anti-4-1BB antibody or an immunogenic fragment of the anti-4-1BB antibody. Other NK cell agonists are known in the art, e.g. see Yea et al. (2015) PNAS, Vol. 112, No. 45, E6158-6165 and Melero et al. (2103) Clin. Cancer Res. 19 (5): 1044-1053, and the disclosed methods are not limited to the anti-4-1BB antibody.

In one embodiment, the plant virus and the NK agonist are in the same composition to be administered simultaneously. In another aspect, the CPMV and the NK agonist are in the different compositions to be administered sequentially, the virus first, or alternatively the NK cell agonist first.

Also provided are combinations or compositions further comprising a carrier. In one aspect, the carrier is a pharmaceutically acceptable carrier.

In another embodiment, the composition or combination of this disclosure comprise a plurality of plant virus that may be the same or different from each other. In another embodiment, the combination or compositions comprise a plurality of NK cell agonists that may be the same or different from each other.

In a yet further aspect, the composition or combination further comprising an additional therapeutic agent.

The combinations or compositions can be used therapeutically, e.g., in a method for inducing an immune response in a subject in need thereof by administering to the subject the composition or combination, alone or in combination with an additional therapeutic agent. In one aspect the combination or compositions improve recruited NK cell function. The combinations or compositions can also be used to treat cancer in a subject in need thereof, comprising, or consisting essentially of, or consisting of administering to the subject the composition or combination alone or in combination with an additional therapeutic agent or tumor resection.

Non-limiting examples of cancers are selected from melanoma, breast cancer, prostate cancer, lung cancer, ovarian cancer, skin cancer, bladder cancer, pancreatic cancer, gastric cancer, esophageal cancer, colon cancer, head and neck cancer, brain cancer, glioma, cervical cancer, hepatocellular cancer, or thyroid cancer. The cancer can be a primary or a metastatic cancer. In one aspect, the cancer is metastatic or primary lung cancer, ovarian cancer, colon cancer, head and neck cancer, melanoma or breast cancer. In another embodiment, the cancer is metastatic melanoma or metastatic colon cancer.

The methods are useful to treat mammals, such as bovines, canines, felines, equines, rats, mice and humans. They can be used in animal models to test additional therapies, or for the treatment of pets and human patients. Any convenient mode of administration can be use, such as for example, intravenous or intraperitoneal delivery.

The therapy can be used to achieve any number of clinical outcomes, e.g., one or more of: inhibiting metastatic potential of the cancer; reduction in tumor size; a reduction in tumor burden, longer progression free survival and longer overall survival of the subject.

Further provided herein is a kit comprising one or more of the composition or combination as disclosed herein and optional instructions for use.

### BRIEF DESCRIPTION OF THE FIGURES

FIGS. 1A-1E: Isolation of CPMV yields monodisperse nanoparticles. (FIG. 1A) UV-Vis spectroscopy spectrum of purified CPMV. (FIG. 1B) Native agarose gel (0.8% w/v) of intact CPMV stained with nucleic acid stain GelRed and protein stain Coomassie blue (CB). Denaturing SDS-PAGE (4-12%) of the coat proteins stained with Coomassie blue identifies the small coat protein (S-CP) of approximately 24 kDa and large coat protein (L-CP) of approximately 42 kDa. (CB). (FIG. 1C) FPLC, (FIG. 1D) DLS, and (FIG. 1E) TEM of purified CPMV (negatively stained with 2% (w/v) uranyl acetate).

FIGS. 2A-2D: Dual therapy with CPMV and anti-4-1BB leads to significantly decreased tumor burden and improved survival in a CT-26 model of colon carcinomatosis. (FIG. 2A) Schematic outline of treatment regimen for PBS (n=5), CPMV (n=7), anti-4-1BB (n=8), and CPMV+anti-4-1BB (n=8) groups in the CT-26-Luc disseminated intraperitoneal colon cancer model. (FIG. 2B) IVIS of PBS, CPMV, anti-4-1BB, and CPMV+anti-4-1BB treatment groups at 7 days post-tumor challenge and following 1 week of treatment at 14 days. (FIG. 2C) Relative percentage weight change and relative abdominal circumference of treatment groups. (FIG. 2D) Survival of treatment groups was plotted and statistical analysis was performed using a Mantel-Cox test (*p<0.05, **p<0.01, ***p<0.005, ****p<0.001). There was a median survival of 21 days (PBS), 24 days (CPMV), 24 days (anti-4-1BB), and indeterminate (CPMV+anti-4-1BB). Data are combined from two independent experimental repeats.

FIGS. 3A-3C: CPMV and anti-4-1BB monotherapy groups demonstrate distinct subgroups of responders and non-responders to treatment in a CT-26 model of colon carcinomatosis. Survival of treatment groups was plotted and statistical analysis was performed using a Mantel-Cox test (*p<0.05, **p<0.01). (FIG. 3A) In the CPMV monotherapy group, out 8 mice there were 4 responders and 4 non-responders. CPMV non-responders demonstrated similar survival compared to PBS control (PBS vs. CPMV non-responders, p=0.184). There was a median survival of 21 days for PBS, 24 days for CPMV, and indeterminate for CPMV+anti-4-1BB groups. For the CPMV subgroups, there was a median survival of 22.5 days for CPMV non-responders and 41 days for CPMV responders. Data are combined from two independent experimental repeats. (FIG. 3B) In the anti-4-1BB monotherapy group, out of 8 mice there were 3 responders and 5 non-responders. For the anti-4-1BB subgroups, there was a median survival of 21 days for anti-4-1BB non-responders and indeterminate for anti-4-1BB responders. Data are combined from two independent experimental repeats. (FIG. 3C) Tumor re-challenge in CPMV+anti-4-1BB treated survivors and naïve BALB/c mice using a subcutaneous model. Tumors were measured with calipers and volumes were estimated as [length×(short width)²]/2. There was a median survival of 21 days (naïve BALB/c, n=8) vs. indeterminate (CPMV+anti-4-1BB survivors, n=8). Statistical analysis was performed using a Mantel-Cox test (***p<0.005).

FIGS. 4A-4C. Dual therapy with CPMV and anti-4-1BB leads to significantly decreased tumor burden and improved survival in a B16F10 model of dermal melanoma. (FIG. 4A) Schematic outline of treatment regimen for PBS (n=9), CPMV (n=10), anti-4-1BB (n=9), and CPMV+anti-4-1BB (n=11) groups in the B16F10 dermal melanoma model. Treatments began when tumors reached ˜40 mm³ and endpoint was defined as tumors exceeding 1500 mm³. (FIG. 4B) Estimated tumor volume as calculated by volume=[(short length)²×(long length)]/2. (FIG. 4C) Survival of treatment groups was plotted and statistical analysis was performed using a Mantel-Cox test (*p<0.05, **p<0.01, ***p<0.005). There was a median survival of 21 days (PBS), 27 days (CPMV), 21 days (anti-4-1BB), and indeterminate (CPMV+anti-4-1BB). Data are combined from two independent experimental repeats.

FIGS. 5A-5B: CPMV monotherapy and CPMV+anti-4-1BB dual therapy groups demonstrate distinct subgroups of responders and non-responders to therapy in a B16F10 model of dermal melanoma. Survival of treatment groups was plotted and statistical analysis was performed using a Mantel-Cox test (**p<0.01, ***<0.005). In the CPMV monotherapy group, out of 7 mice there were 3 responders and 4 non-responders. There was a median survival of 21 days for PBS, 27 days for CPMV, and indeterminate for CPMV+anti-4-1BB treated groups. For the CPMV subgroups, there was a median survival of 22 days for CPMV non-responders and indeterminate for CPMV responder subgroups. For the dual therapy CPMV+anti-4-1BB subgroups, there was a median survival of 26.5 days for CPMV+anti-4-1BB responders and indeterminate for CPMV+anti-4-1BB responders. (FIG. 5A) Data are combined from two independent experimental repeats. (FIG. 5B) Tumor re-challenge in CPMV+anti-4-1BB treated survivors and naïve C57Bl/6J mice with median survival of 17 days (naïve C57Bl/6J, n=5)) vs. 21 days (CPMV+anti-4-1BB survivors, n=4). Data are combined from two experimental repeats. Statistical analysis was performed using a Mantel-Cox test (*p<0.05).

FIG. 6: Schematic of treatment response. Treatment with CPMV recruits NK cells to the tumor microenvironment, and monoclonal NK cell agonist anti-4-1BB antibody activates recruited intratumoral NK cells. Together, this leads to immune-mediated tumor destruction. Presentation of antigen to T cells then initiates an adaptive immune response and subsequent immunological memory.

### DETAILED DESCRIPTION

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., virus or agonist of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the phrase “inducing an immune response”, “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells-non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells. Thus, in one aspect, inducing an immune response refers to activating the cellular immune response in a subject after therapy. Methods to determine if an immune response are known in the art, and include assaying for the induction of cytokines, radioimmunoassay (RIA), enzyme-linked immunoabsorbent assay (ELISA), and immunoblotting techniques.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

An “NK cell intends a type of immune cell that has granules (small particles) with enzymes that can kill tumor cells or cells infected with a virus. A natural killer cell is a type of white blood cell, and is also called an NK cell and NK-LGL.

An “NK cell agonist” intends a small molecule, peptide or biologic that augments or activated an NK cell in vitro or in vivo. The NK cell agonist can in one aspect, recognize and bind a stimulatory receptor on one or more NK cells, thereby activating or proliferating NK cells. These can be antibodies or fragments thereof that bind the receptor. A non-limiting example is a 4-1BB antibody or fragment thereof that binds the receptor. NK cell activating receptors are known in the art, examples of such are provided below. Mouse/human: NKG2D (CD314); CD94-NKG2C; NKp46 (NCR1); NKp44 (NCR2) and DNAM-1. Mouse: Ly49D; Ly49H; NKR-P1C; and NKR-P1G. Human: KIR2DL4 (CD158d); KIR2DS1 (CD158h); KIR2DS2 (CD158j); KIR2DS3; KIR2DS4 (CD158i); KIR2DS5 (CD158g); KIR3DS1 (CD158e2); KIR2DL4 (CD158d); NKp30 (NCR3); CD16 (FCGR3, Fc-gamma-III); SLAMF7; SLAMF6; TACTILE; NKp80; CD27; CD94-NKG2C; and CD94-NKG2E. See, e.g., Paul, Sourav, and Girdhari Lal. Frontiers in Immunology 8 (2017): 1124; Liu, Sizhe, et al. J. Hematology & Oncology 14 (2021): 1-17; and Chu, Junfeng, et al. J. of Translational Medicine 20.1 (2022): 1-19.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated. In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

### Modes for Carrying Out the Disclosure

### Plant Virus

In some embodiments, the virus is derived from Cowpea chlorotic mottle virus (CCMV). CCMV is a spherical plant virus that belongs to the Bromovirus genus. Several strains have been identified and include, but not limited to, Car1 (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean(S) (Kuhn, 1968. Phytopathology 58:1441-1442), mild (M) (Kuhn, 1979. Phytopathology 69:621-624), Arkansas (A) (Fulton, et al., 1975. Phytopathology 65:741-742), bean yellow stipple (BYS) (Fulton, et al., 1975. Phytopathology 65:741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72 (9): 768-770).

In some instances, the virus from CCMV comprise, or consists essentially of, or yet further consists of, a plurality of capsid proteins. In some instances, the capsid protein is a wild-type CCMV capsid, optionally expressed by Car1, Car2, type T, soybean(S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, the sequence as set forth in the UniProtKB ID P03601:

MSTVGTGKLTRAQRRAAARKNKRNTRVVQPVIVEPIASGQGKAIK
AWTGYSVSKWTASCAAAEAKVTSAITISLPNELSSERNKQLKVGR
VLLWLGLLPSVSGTVKSCVTETQTTAAASFQVALAVADNSKDVVA
AMYPEAFKGITLEQLTADLTIYLYSSAALTEGDVIVHLEVEHVRP
TFDDSFTPVY
(SEQ ID NO: 1), or an equivalent thereof.

In some cases, the virus from CCMV is prepared by the method as described in Ali et al., “Rapid and efficient purification of Cowpea chlorotic mottle virus by sucrose cushion ultracentrifugation,” Journal of Virological Methods 141:84-86 (2007).

In some embodiments, the virus is or is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the virus from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

MEQNLFALSLDDTSSVRGSLLDTKFAQTRVLLSKAMAGGDVLLDE
YLYDVVNGQDFRATVAFLRTHVITGKIKVTATTNISDNSGCCLML
AINSGVRGKYSTDVYTICSQDSMTWNPGCKKNFSFTFNPNPCGDS
WSAEMISRSRVRMTVICVSGWTLSPTTDVIAKLDWSIVNEKCEPT
IYHLADCQNWLPLNRWMGKLTFPQGVTSEVRRMPLSIGGGAGATQ
AFLANMPNSWISMWRYFRGELHFEVTKMSSPYIKATVTFLIAFGN
LSDAFGFYESFPHRIVQFAEVEEKCTLVFSQQEFVTAWSTQVNPR
TTLEADGCPYLYAIIHDSTTGTISGDFNLGVKLVGIKDFCGIGSN
PGIDGSRLLGAIAQ
(SEQ ID NO: 2), or an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, the sequence as set forth in the UniProtKB ID P03599 (residues 834-1022):

GPVCAEASDVYSPCMIASTPPAPFSDVTAVTFDLINGKITPVGDD
NWNTHIYNPPIMNVLRTAAWKSGTIHVQLNVRGAGVKRADWDGQV
FVYLRQSMNPESYDARTFVISQPGSAMLNFSFDIIGPNSGFEFAE
SPWANQTTWYLECVATNPRQIQQFEVNMRFDPNFRVAGNILMPPF
PLSTETPPL
(SEQ ID NO: 3), or an equivalent thereof.

In some embodiments, the virus is derived from Physalis mottle virus (PhMV). PhMV is a single stranded RNA virus that belongs to the genus Tymovirus. In some instances, the virus or VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, the sequence as set forth in the UniProtKB ID P36351:

MDSSEVVKVKQASIPAPGSILSQPNTEQSPAIVLPFQFEATTFGT
AETAAQVSLQTADPITKLTAPYRHAQIVECKAILTPTDLAVSNPL
TVYLAWVPANSPATPTQILRVYGGQSFVLGGAISAAKTIEVPLNL
DSVNRMLKDSVTYTDTPKLLAYSRAPTNPSKIPTASIQISGRIRL
SKPMLIAN
(SEQ ID NO: 4), or an equivalent thereof.

In some embodiments, the virus is derived from Sesbania mosaic virus (SeMV). SeMV is a positive stranded RNA virus that belongs to the genus Sobemovirus. In some instances, the virus or VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprise, or consists essentially of, or yet further consists of, the sequence as set forth in the UniProtKB ID Q9EB06:

MAKRLSKQQLAKAIANTLETPPQPKAGRRRNRRRQRSAVQQLQPT
QAGISMAPSAQGAMVRIRNPAVSSSRGGITVLTHSELSAEIGVTD
SIVVSSELVMPYTVGTWLRGVAANWSKYSWLSVRYTYIPSCPSST
AGSIHMGFQYDMADTVPVSVNQLSNLRGYVSGQVWSGSAGLCFIN
GTRCSDTSTAISTTLDVSKLGKKWYPYKTSADYATAVGVDVNIAT
PLVPARLVIALLDGSSSTAVAAGRIYCTYTIQMIEPTASALNN
(SEQ ID NO: 5), or an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid. \Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a virus described herein further comprise, or consists essentially of, or yet further consists of, a label or a tag, e.g., such as a detectable label. A detectable label can be attached to, e.g., to the surface of a virus or VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the virus or NK cell agonist. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

### Compositions

In another aspect, provided herein is the combination or composition that further comprises at least one carrier, such as a pharmaceutically acceptable carrier or excipient. In one aspect, the composition further comprises a preservative or stabilizer.

In one embodiment, this technology relates to a composition comprising a combination of virus and NK agonist in one or composition or in separate compositions, so that they can be administered sequentially and a carrier.

Compositions, including pharmaceutical compositions comprising, consisting essentially of, or consisting of the virus and/or NK cell agonist can be in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, subcutaneous, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration or the combinations of virus and/or NK cell agonist can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the combinations or compositions are administered to a subject suffering from a condition as disclosed herein, such as a mammal or a human, either alone or as part of a pharmaceutically acceptable formulation, once a week, once a day, twice a day, three times a day, or four times a day, or even more frequently.

Administration of the composition or combination alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Therapeutic Methods

Further disclosed herein are methods for inducing an immune response in a subject consisting essentially of, or yet further consisting of the combination or composition as disclosed herein. In one aspect, the composition or combination comprises or consists essentially of, or consists of a plant virus selected from Cowpea chlorotic mottle virus (CCMV), Cowpea mosaic virus (CPMV), Physalis mottle virus (PhMV), and Sesbania mosaic virus (SeMV), and a natural killer (NK) cell agonist. In one aspect, the composition or combination comprises, or consists essentially of, or consists of a Cowpea mosaic virus (CPMV) and a natural killer (NK) cell agonist. NK cell agonist are known in the art and described herein. In one aspect, the NK cell agonist comprises an anti-4-1BB antibody or an immunogenic fragment of the antibody. In another aspect, the immunogenic fragment of the antibody is selected from Fab, Fab′, F(ab′)₂, Fv, scFv, dsFv, or Fd fragment.

In one aspect the combination or compositions induce an immune response by improving recruited NK cell function.

In one aspect, the subject is a mammal or a human. In one aspect, the administering comprises intravenous, subcutaneous, intratumoral, or intraperitoneal delivery.

In one embodiment, the plant virus and the NK agonist are administered or delivered in the same composition. In another embodiment, the plant virus and the NK agonist are in the different compositions. These can be administered with a carrier, such as a pharmaceutically acceptable carrier.

The method can be practiced with a plurality of plant virus, which can be the same or different from each other. Alternatively, the plurality of NK cell agonists that may be the same or different from each other.

The method can further comprise administration of an additional therapeutic agent, such as for example, those known in the art or alternatively as described herein.

Also provided herein is a method for treating cancer in a subject in need thereof, comprising administering to the subject the composition or combination as described herein. In one aspect, the composition or combination comprises or consists essentially of, or consists of a plant virus selected from Cowpea chlorotic mottle virus (CCMV), Cowpea mosaic virus (CPMV), Physalis mottle virus (PhMV), and Sesbania mosaic virus (SeMV), and a natural killer (NK) cell agonist. In one aspect, the composition or combination comprises, or consists essentially of, or consists of a Cowpea mosaic virus (CPMV) and a natural killer (NK) cell agonist. NK cell agonist are known in the art and described herein. In one aspect, the NK cell agonist comprises an anti-4-1BB antibody or an immunogenic fragment of the antibody. In another aspect, the immunogenic fragment of the antibody is selected from Fab, Fab′, F(ab′)₂, Fv, scFv, dsFv, or Fd fragment.

In one embodiment, the plant virus and the NK agonist are administered or delivered in the same composition. In another embodiment, the plant virus and the NK agonist are in the different compositions. These can be administered with a carrier, such as a pharmaceutically acceptable carrier.

The method can be practiced with a plurality of plant virus, which can be the same or different from each other. Alternatively, the plurality of NK cell agonists that may be the same or different from each other.

The method can further comprise administration of an additional therapeutic agent, such as for example, those known in the art or alternatively as described herein.

In one aspect, the cancer is a blood cancer or a solid tumor, such as for example, a cancer is selected from melanoma, breast cancer, prostate cancer, lung cancer, ovarian cancer, skin cancer, bladder cancer, pancreatic cancer, gastric cancer, esophageal cancer, head and neck, colon cancer, brain cancer, glioma, cervical cancer, hepatocellular cancer, or thyroid cancer. The cancer can be a primary or a metastatic cancer. In a further aspect, the cancer is metastatic or primary lung cancer, ovarian cancer, colon cancer, melanoma or breast cancer or alternatively, wherein the cancer is metastatic melanoma or metastatic colon cancer.

The method can be combined with a different cancer therapy or tumor resection.

The method provides a treatment from one or more of: inhibiting metastatic potential of the cancer; reduction in tumor size; a reduction in tumor burden, longer progression free survival and longer overall survival of the subject.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, head and neck, colon cancer, brain cancer, glioma, cervical cancer, hepatocellular cancer, or thyroid cancer. In some embodiments, the cancer is primary or metastatic cancer. In some embodiments, the cancer is metastatic or primary lung cancer or breast cancer. In some embodiments, the cancer metastatic melanoma or metastatic triple negative breast cancer. In some embodiments, the cancer is a primary or metastatic cancer in lung.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, or inhalation. In some embodiments, administering is intravenous.

The methods and compositions disclosed herein may further comprise or alternatively consist essentially of, or yet further consists of administering to the subject an anti-tumor therapy other than the virus and NK cell agonist disclosed herein. In some embodiments, anti-tumor therapy may include different cancer therapy or tumor resection. The additional therapeutic can be combined in the same composition or separately administered.

In some embodiments, the combination or composition are provided to prevent the symptoms of cancer from occurring in a subject that is predisposed or does not yet display symptoms of the cancer.

In some embodiments, the combination or composition disclosed herein may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, the combination or composition are administered to the subject at least 1, 2, 3, 4, 5, 6, 7, 8, 9, or 10 times a day. In some embodiments, any of polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, the combination or composition disclosed herein are administered to the subject at least every 1, 2, 3, 4, 5, 6, 7, 8, 9, or 10 days. In some embodiments, the combination or composition disclosed herein are administered to the subject at least every 1, 2, 3, 4, 5, 6, 7, 8, 9, 10, 11, 12, 13, or 14 weeks. In some embodiments, the combination or composition disclosed herein are administered to the subject for a period of at least 1, 2, 3, 4, 5, 6, 7, 8, 9, or 10 days. In some embodiments, the combination or composition disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, the combination or composition disclosed herein are administered to the subject for a period of at least 1, 2, 3, 4, 5, 6, 7, 8, 9, 10, 12, 13, 14, 15, 16, 17, 18, 19, or 20 months.

In some embodiments, the methods, the combinations or compositions provided herein, comprising, or alternatively consisting essentially of, or yet further consisting inhibiting metastatic potential of the cancer, reduction in tumor size, a reduction in tumor burden, longer progression free survival, or longer overall survival of the subject.

In one aspect, the methods, combination or compositions further comprise administration of an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the combination or composition with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACAR, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A: U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the composition or combination is administered in combination with a radiation therapy.

### Kits

In one particular aspect, the present disclosure provides kits for performing the methods of this disclosure as well as instructions for carrying out the methods of the present disclosure. The kit comprises, or alternatively consists essentially of, or yet further consists of one or more of the combination or composition and instructions for use. In a further aspect, the instruction for use provide directions to conduct any of the methods disclosed herein.

The kits are useful for detecting the presence of cancer such as lung cancer in a biological sample e.g., any bodily fluid including, but not limited to, e.g., sputum, serum, plasma, lymph, cystic fluid, urine, stool, cerebrospinal fluid, ascitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Experimental

In tumor mouse models with intraperitoneal (i.p.) disseminated metastatic tumors, i.p. treatment with CPMV results in up to 2-fold increase in recruitment of NK cells to the tumor microenvironment within 6 hours after treatment and approximately 10-fold increase at 48 hours^{6,10} NK cells are derived from common lymphoid progenitors but function as innate immune cells, and have the unique ability to recognize and destroy virally infected or transformed cells without co-stimulation, releasing foreign antigens to initiate an adaptive immune response^{4,14-16}. NK cells may be activated through stimulation of several receptors, including 4-1BB (CD137)¹⁷⁻¹⁹. However, exploiting NK cells in cancer immunotherapy is met by two major challenges: 1) recruiting NK cells to the tumor microenvironment, and 2) stimulating NK cells to overcome the immunosuppressive hurdle of the tumor microenvironment to enact NK cell function. Typically, NK cells are able to recognize virally infected or transformed cancer cells by their lack of MHC Class I. However, within the tumor microenvironment, while cancer cells tend to have decreased expression of MHC Class I (which would make them recognizable by NK cells), the cancer cells can secrete a soluble form of MHC Class I which essentially saturates receptors on NK cells and suppresses them from functioning appropriately⁴. This overwhelming suppression outbalances stimulatory molecules and enables the tumor to evade immunological attack from NK cells. Nevertheless, stimulating intratumoral NK cells and enabling them to respond to transformed cells can prove to be advantageous in initiating an immunological response to cancer^{4,20-23}.

NK cells may be stimulated via a variety of mechanisms such as stimulation with cytokines, e.g. IL-12 or stimulation of activation receptors, e.g. 4-1BB or NKG2D. Data indicate treatment with anti-4-1BB agonist to be NK cell-dependent and effective in treating murine tumor models^{4,20-23}; therefore, Applicant selected anti-4-1BB as a therapeutic approach to stimulate NK cells. It is off note though that 4-1BB is also expressed on lymphocytes, neutrophils, and macrophages, therefore activation of immune cells may occur more broadly. In NK cells, agonism of the 4-1BB receptor activates NFkB, ERK, and P38-MAP kinase pathways¹⁹, thus priming recruited NK cells to escape the immunosuppressive state and to become activated, and execute cytotoxic functions on tumor cells²³.

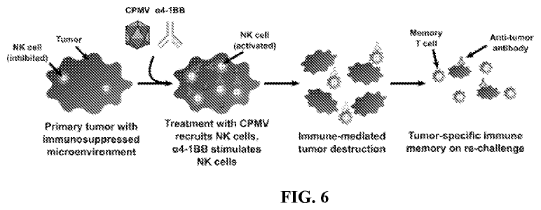

Thus, as illustrated graphically in FIG. 6, Applicant hypothesized that treatment with CPMV 4-1BB would facilitate direct cytotoxicity of tumor cells, enhance innate immune cell processing and presentation of tumor antigens to the adaptive immune system, and improve in situ cancer vaccination efficacy.

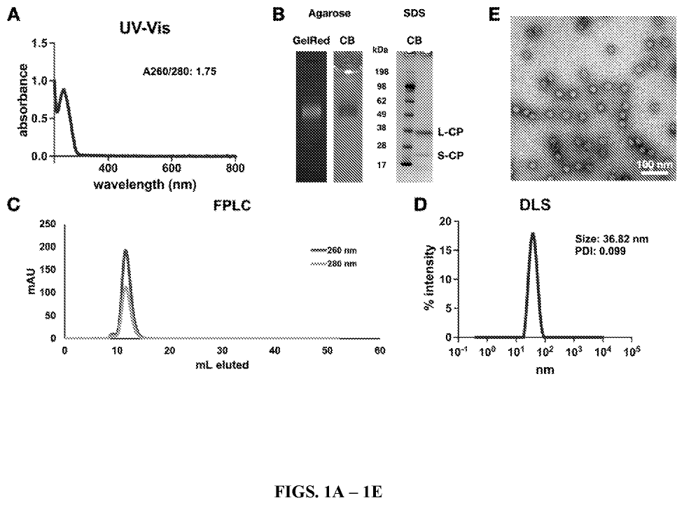

CPMV was produced in black-eyed pea No. 5 plants and purified CPMV was characterized using several techniques to ensure quality control. UV-Vis was used to determine the concentration of purified CPMV and the A260/280 ratio of 1.75 also indicates that pure CPMV was obtained (FIG. 1A). CPMV was further characterized using native agarose and denaturing SDS-PAGE to characterize intact particles and coat proteins, respectively (FIG. 1B). On the native agarose gel, RNA co-migrates with the capsid indicating presence of intact CPMV particles. SDS-PAGE reveals the small (S-CP, ˜24 kDa) and large (L-CP, ˜42 kDa) coat protein subunits. Together, both agarose and SDS-PAGE methods indicate that pure CPMV preparations were obtained in the absence of free nucleic acids or protein contaminants. Fast protein liquid chromatography (FPLC) demonstrated uniform CPMV preparations with an elution of particles at 11.6 mL, characteristic for CPMV (FIG. 1C); the 260 nm (RNA) and 280 nm (protein) peak overlap, again demonstrating that intact CPMV was eluted from the column. Additionally, aggregation, disassembly or contaminants were not apparent. This was also consistent with dynamic light scattering (DLS) showing a uniform population of particles with an average particle size of 36.82 nm (PDI 0.099) (FIG. 1D). Monodisperse CPMV particles were also imaged by transmission electron microscopy (TEM) (FIG. 1E). Together, this data demonstrates pure preparations of monodisperse and intact CPMV were obtained.

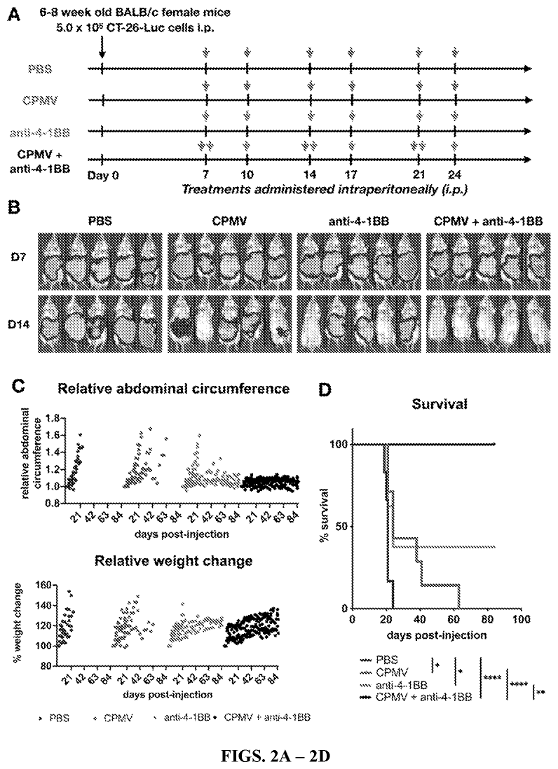

To test the efficacy of combined CPMV and NK cell agonist immunotherapy, Applicant first utilized the CT-26 model of colon carcinomatosis. Intraperitoneal (i.p.) disseminated tumors were established in BALB/c mice as previously described^{24,25}. One week after tumor challenge, mice were assigned to one of four treatment groups: PBS vehicle control, CPMV, anti-4-1BB NK cell agonist (BioXCell, clone LOB12.3), or dual therapy CPMV+anti-4-1BB (n=6-8 per group). Treatments were initiated 7 days after tumor challenge and continued for 3 weeks. CPMV treatments (50 μg i.p.) were administered once weekly, anti-4-1BB treatments (5 μg i.p.) were administered twice weekly, and PBS vehicular control was administered twice weekly (see FIG. 2A). Using the CT-26 i.p. tumor mouse model, Applicant previously demonstrated potent and durable efficacy of CPMV at three weekly i.p. doses of 100 μg CPMV as a monotherapy. Therefore, to assess the efficacy as combination with NK agonist therapy, Applicant lowered the dose to 50 μg. Tumor burden was followed using weight, abdominal circumference, and IVIS imaging of Luc-labeled CT-26 cells. After 1 week of treatment, mice treated with CPMV or anti-4-1BB alone demonstrated decreased luciferase bioluminescence compared to PBS vehicular controls. More importantly, mice treated with CPMV+anti-4-1BB dual immunotherapy demonstrated reduction of luciferase bioluminescence to near-background levels (FIG. 2B). The negative control group of PBS-treated mice demonstrated a rapid increase in weight, abdominal circumference, and mortality (FIGS. 2C, 2D). Of mice receiving monotherapy treatment of CPMV or anti-4-1BB alone, there was significantly improved survival compared to the PBS control group (CPMV 24 days vs. PBS 21 days, p<0.01; anti-4-1BB 24 days vs. PBS 21 days, p<0.05). Impressively, mice treated with CPMV+anti-4-1BB dual therapy showed no signs of disease—while they gained physiological weight there was no change in abdominal circumference (a measure of tumor burden). Most importantly, all mice treated with the dual therapy regimen remained alive at the end of the study.

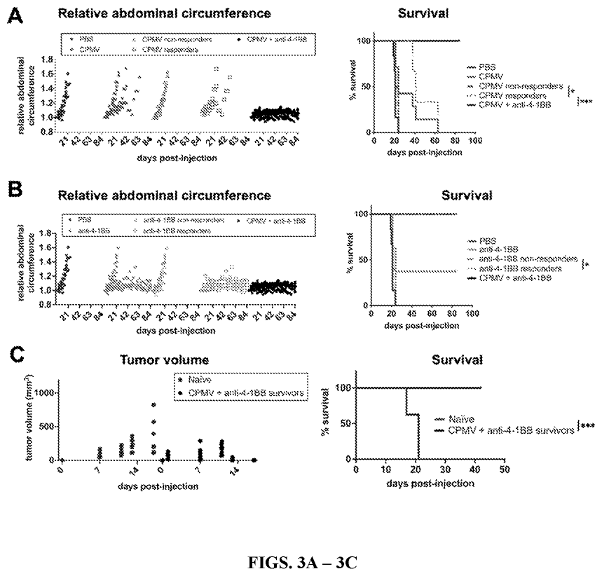

Assessment of the raw data demonstrated two distinct groups of responders and non-responders in CPMV and anti-4-1BB monotherapy groups. 4 of 7 CPMV and 5 of 8 anti-4-1BB monotherapy treated mice were non-responders and demonstrated rapid increase in weight, abdominal circumference parameters, and early mortality (as per humane endpoints) comparable to those treated with PBS (FIGS. 3A, 3B). In contrast, 3 of 7 CPMV and 3 of 8 anti-4-1BB monotherapy mice demonstrated at least partial response with delayed increases in weight and abdominal circumference parameters and improved survival compared to the respective non-responder subgroup. CPMV responders had a median survival of 41 days compared to CPMV non-responders with a median survival of 22.5 days (CPMV responders vs. CPMV non-responders, p<0.05). Likewise, anti-4-1BB responders survived through the entirety of the study while anti-4-1BB non-responders had a median survival of 21 days (anti-4-1BB responders vs. anti-4-1BB non-responders, p<0.05). CPMV responders did ultimately succumb to tumor burden, which may be partially due to the intentional sub-therapeutic dosing of CPMV used in these experiments. Interestingly, as anti-4-1BB responders demonstrated no signs of residual disease following treatment, this subgroup was comparable to the dual-therapy CPMV+anti-4-1BB group with no significant difference in mortality (anti-4-1BB responders vs. anti-4-1BB, p=0.094). This distinction of responders and non-responders highlights that while monotherapy treatments may be partially effective on their own, the synergistic effect of dual therapy with CPMV and anti-4-1BB remains the most effective treatment to reduce tumor burden and improve survival.

After at least 30 days of tumor clearance, survivors (and naïve BALB/c control mice) were re-challenged to determine whether the treatment induced immunological memory. This time, Applicant challenged mice with CT-26 cells s.c. and monitored tumor volume. Naïve mice demonstrated growth of CT-26 subcutaneous tumors and average mortality at 21 days (FIG. 3C). In contrast, subcutaneous CT-26 tumors of dual-therapy-treated mice initially demonstrated mild tumor growth followed by resolution of tumor burden. All dual-therapy-treated mice remained alive after 42 days following tumor re-challenge, demonstrating that the CPMV+anti-4-1BB dual therapy elicits potent and durable anti-tumor immunity.

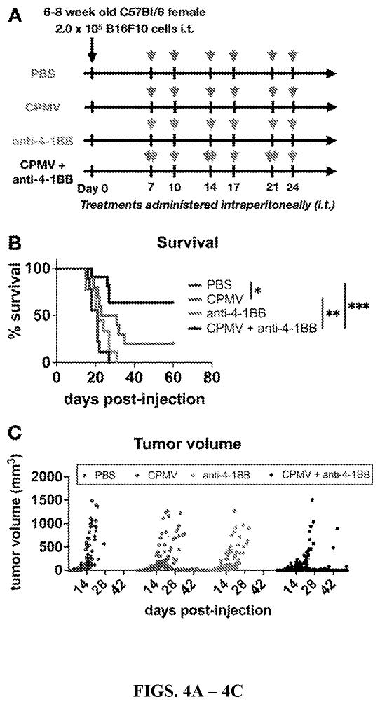

A valuable feature of CPMV cancer immunotherapy is that it is a single biologic that has the potential to induce a personalized tumor-specific immunotherapy approach. That is, the CPMV in situ vaccine is not tumor specific or tailored to a certain tumor type, but rather CPMV has demonstrated potent efficacy in multiple tumor types through innate immune stimulation and reversion of immunosuppression by turning cold into hot tumors^{9,10}. Therefore, to validate the robustness of the CPMV+NK cell agonist dual immunotherapy, Applicant next evaluated the efficacy of the dual-pronged therapeutic approach using a dermal melanoma mouse model using B16F10 cells in female C57Bl/6 mice. Applicant established dermal melanoma tumors as previously described^{24,26,27} (n=9-11 per group) and began treatments when tumor volumes reached approximately 40 mm³, approximately 7 days after tumor challenge and continued for 3 weeks (FIG. 4A). To assess disease burden and efficacy, Applicant monitored tumor volume and mortality. CPMV treatments (50 μg intratumorally (i.t.)) were administered once weekly, anti-4-1BB treatments (5 μg i.t.) were administered twice weekly, and PBS vehicular control was administered twice weekly. Again, similar to the CT-26 model, Applicant designed the treatment plan for the CPMV monotherapy arm to be suboptimal from Applicant's previous work (where Applicant showed 100 μg CPMV i.t. weekly to be an effective dose) to allow for a possible synergistic effect of the dual therapy to be able to be detected.

Mice treated with PBS demonstrated rapid tumor growth and an average mortality of 21 days (FIGS. 4B, 4C). Additionally, mice treated with anti-4-1BB alone demonstrated similar average mortality of 21 days (PBS vs. anti-4-1BB, p=0.31). As a group, CPMV mice demonstrated significantly improved survival compared to PBS-treated mice with an average mortality of 27 days (PBS vs. CPMV, p<0.05). In the dual-therapy CPMV+anti-4-1BB group there were 7 of 11 mice surviving by the end of the study, demonstrating significantly improved survival compared to PBS-treated mice (PBS vs. CPMV+anti-4-1BB, p<0.01) albeit statistical significance between CPMV+anti-4-1BB and CPMV alone was not observed (p=0.053).

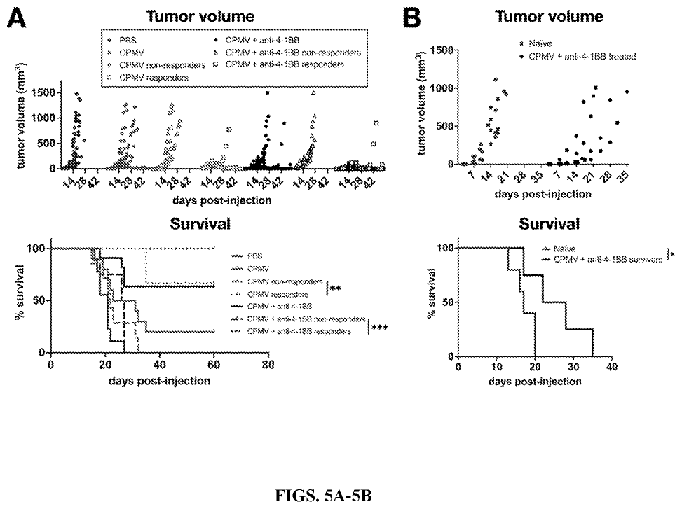

Similar to the CT-26 model, there were responders and non-responders in those treated with CPMV alone (FIG. 5). In the CPMV monotherapy group, 3 of 10 mice demonstrated delayed tumor growth, with 1 of the responders eventually succumbing to the tumor burden following completion of treatment as outlined in FIG. 4A. In contrast, 7 of 10 CPMV monotherapy treated mice did not demonstrate any substantial response to treatment with a median survival of 22 days, similar to a median survival of 21 for PBS-treated mice (PBS vs. CPMV non-responders, p=0.142). In the CPMV+anti-4-1BB dual therapy group, 7 of 11 animals demonstrated robust response to therapy and total or near-total resolution of their dermal melanoma tumors (FIG. 5). In contrast, 4 of the 11 mice treated with dual therapy did not demonstrate any substantial response to treatment and had a median survival of 26.5 days, not significantly different from the median survival of PBS controls with a median survival of 21 days. There was no significant difference in the median survival of CPMV responder and CPMV+anti-4-1BB responder subgroups (p=0.127); however, detection of any statistical difference between these two subgroups may be limited by the decreased statistical power in comparing these smaller experimental subgroups.

After at least 30 days of tumor clearance, Applicant then re-challenged survivors treated with CPMV+anti-4-1BB and naïve C57Bl/6 mice with B16F10 intradermal melanoma injected on the opposite flank and monitored tumor volume and overall mortality (FIG. 5B). Compared to naïve mice, the previously treated CPMV+anti-4-1BB survivors demonstrated delayed tumor growth and significantly increased survival (21 days vs 17 days, p=0.042, FIG. 5B), again demonstrating that CPMV immunotherapy induces potent and long-lasting anti-tumor immunity and immunological memory.

In summary, Applicant demonstrated the efficacy of dual therapy of CPMV and anti-4-1BB in cancer in situ vaccination. In the CT-26-Luc model of disseminated colon carcinomatosis, CPMV and anti-4-1BB dual therapy demonstrated significantly improved survival compared to PBS controls and both CPMV and anti-4-1BB monotherapy groups. Remarkably, in the CT-26-Luc model all mice receiving CPMV+anti-4-1BB dual therapy demonstrated rapid regression of tumor burden, and tumors were not detected throughout the remainder of the study. Furthermore, re-challenge with subcutaneous CT-26-Luc cells demonstrated immunological memory and elimination of tumor cells. Together, this data show that CPMV and anti-4-1BB dual therapy is highly effective at reducing tumor burden, improving survival, and eliciting a potent immunological memory response.

In the B16F10 dermal melanoma model, dual therapy with CPMV and anti-4-1BB demonstrated significantly improved survival compared to PBS control and anti-4-1BB monotherapy groups, with a trend toward significant improvement compared to CPMV monotherapy. Re-challenge with intradermal melanoma tumors on the contralateral flanks demonstrated decreased tumor growth and improved survival compared to naïve animals, suggesting tumor-specific immunological memory. Future studies will focus on dosing and safety assessment of mono- and combination therapy arms.

Treatment with anti-4-1BB alone or CPMV+anti-4-1BB dual therapy was less effective in the B16F10 dermal melanoma model compared to the CT-26-Luc model of colon carcinomatosis. This may be due to the differences in the tumor microenvironment. In the intraperitoneal CT-26 model, treatments diffuse and spread throughout the peritoneal cavity enabling interaction with tumor cells which may also enable more efficient recruitment of NK cells by CPMV. In contrast, solid dermal B16F10 tumors may be more restrictive to CPMV diffusion, CPMV-mediated NK cell recruitment, and/or diffusion of the anti-4-1BB antibody. It is possible this may be overcome with modification, such as using multiple injection sites or delivery of therapeutics via microneedle patches. Indeed, Applicant have previously demonstrated that efficacy of immunotherapy can be improved in solid tumors through microneedle patch delivery²⁸.

Ultimately, this work demonstrates synergy between CPMV and anti-4-1BB immunotherapy treatments.

Oncolytic cancer immunotherapies are undergoing development and several are being used clinically. While the plant virus in situ vaccine and the oncolytic approach are conceptually distinct (oncolytic viruses target and kill tumor cells directly, while the plant virus targets innate immune cells to active cell killing function by the immune system), data suggest that oncolytic viruses have the potential to be further modified to further recruit and activate NK cells^{29,30}. For example, this may be accomplished by creating recombinant oncolytic viruses expressing various cytokines promoting NK cell activation, such as IL-2^{31,32} and IL-15³³. While promising, such avenues require further recombinant engineering and optimization whereas CPMV is inherently capable of recruiting NK cells to the tumor microenvironment. Given the unique potency and potential to combine with NK agonists as well as other immunotherapies such as checkpoint inhibitors³⁴, chemotherapeutic agents³⁵, and radiation³⁶, CPMV immunotherapy makes an attractive platform technology for multimodal cancer immunotherapy.

As Applicant move forward to the potential use of CPMV in human patients, biosafety profiles and production methods must be considered. As CPMV is not inherently infectious toward humans, Applicant anticipate CPMV will have an improved biosafety profile for both the patient and the administering healthcare professionals compared to oncolytic viral treatments. Additionally, another favorable attribute is its high yielding production through molecular farming in plants; 1 plant yields ˜1 mg of pure CPMV³⁷ which is sufficient to complete the treatment schedule for 1 canine patient³⁸. As canine patients present with tumors comparable in size to human tumors, Applicant project the human dose to be equivalent to the canine dose. However, clinical trials remain necessary to determine the dose and safety profile of CPMV for use in human patients.

Being able to recruit NK cells to the tumor microenvironment and take advantage of their innate cytolytic functions in cancer immunotherapies has been a challenge for many years. The ability to enable NK cells to function appropriately against tumor cells despite strong immunosuppressive signals within the tumor microenvironment would enable improved processing of tumor antigens and a robust anti-tumor immunologic response. This work shows that a dual-therapy approach using CPMV and a NK cell agonist improves tumor-specific immunological response and improved survival. This approach may be essential to fully utilize NK cell capabilities in the development of new and potent cancer immunotherapies.

### Materials and Methods

### Production and Characterization of CPMV

CPMV was produced in black-eyed pea No. 5 (Vigna unguiculata) plants using methods previously described (H. S. Leong et al., Nat. Protoc. 5, 2010). Leaves were dusted with carborundum and inoculated with 0.1 mg/mL CPMV and harvested after 12-14 days after viral propagation. Leaves were frozen at −80° C. or directly processed for CPMV purification using chloroform-butanal extraction, PEG precipitation and isopycnic ultracentrifugation as previously established¹. Following purification, viral concentration was determined using UV-vis spectroscopy (§ 260 nm=8.1 mL/mg*cm). Particle integrity was verified using size exclusion chromatography (SEC) using a ÄKTA Explorer chromatography system (GE Healthcare) and Superose6 column, transmission electron microscopy (TEM) using FCF400-CU 400-mesh copper grids (Electron Microscopy Sciences) and a FEI Tecnai G2 Spirit TEM at 80 kV, as well as dynamic light scattering (Zetasizer Nano ZSP, Malvern). SDS-PAGE using 4-12% NuPAGE gels (Invitrogen) in 1×MOPS buffer and 0.8% (w/v) agarose gels in 1×TBE were used to confirm CPMV integrity and purity using GelRed nucleic acid staining and/or Coomassie blue staining.

### Cell Culture

CT-26-Luc cells were cultured in RPMI with 10% (v/v) fetal bovine serum and 1% (v/v) 100× penicillin/streptomycin. B16F10 cells were cultured in DMEM with 10% (v/v) fetal bovine serum and 1% (v/v) 100× penicillin/streptomycin. Cells were counted using a hemocytometer and washed in sterile 1×PBS prior to use for in vivo studies.

Ct-26-Luc Colon Cancer and B16F10 Dermal Melanoma Studies Animal studies were performed using University of California, San Diego (UCSD) Institutional Animal Care Use Committee (IACUC)-approved protocols.

### CT-26-Luc Colon Cancer

6-8 week-old female BALB/c mice (Jackson Labs) were injected intraperitoneally with 5×10⁵ cells of luciferase-positive murine colon cancer cell line CT-26-Luc in sterile PBS. Tumor burden was monitored using the Perkin Elmer IVIS Spectrum, in which mice were injected with 150 μL of 15 mg/mL luciferin and imaged 5 minutes post-injection with a 1-minute exposure time. Total luminescence was determined using Living Image software and data was graphed as total counts per mouse. Treatment was initiated 7 days following initial tumor challenge and administered as 50 μg CPMV weekly (day 7, 14, and 21), 5 μg anti-4-1BB (BioXCell, clone LOB12.3) twice weekly (day 7, 10, 14, 17, 21, and 24), combined weekly 50 μg CPMV and twice weekly 5 μg anti-4-1BB, or twice-weekly PBS as vehicle control (n=6-8 per group). Health metrics were monitored at least twice weekly including mouse weight and abdominal circumference. Animals were humanely euthanized when excessive tumor burden or health metrics were observed including increase in weight by 50% or increase in abdominal circumference by 60%.

Survivors following 80-90 days from primary tumor challenge and at least 30 days from tumor clearance were re-challenged with 2.5×10⁵ CT-26 cells injected subcutaneously (s.c.). An additional group of naïve age-matched BALB/c female mice were used as controls. 2×10⁴ cells were mixed 1:1 by volume with Matrigel and injected s.c. over the posterior flank. Tumors were measured using digital calipers and tumor volume was estimated as [length×(short width)²]/2. Animals were humanely euthanized if tumor volumes exceeded 1500 mm³ or weight loss of 20%.

### B16F10 Dermal Melanoma

6-8 week-old female C57B16/J mice (Jackson Labs) were injected intradermally over the posterior flank with 2.5×10⁵ B16F10 murine dermal melanoma cells in sterile PBS (n=9-11 per group). Tumor burden was monitored using digital calipers and tumor volume was estimated as detailed above. Animals were humanely euthanized if tumor volumes exceeded 1500 mm³ or weight loss of 20%.

Survivors following 80-90 days from primary tumor challenge and at least 30 days from tumor clearance were re-challenged with 2.5×10⁵ B16F10 cells in sterile PBS injected intradermally over the opposite posterior flank. An additional group of naïve age-matched C57Bl/6J female mice were used as controls. 2.5×10⁵ B16F10 murine dermal melanoma cells in sterile PBS. Tumor size was monitored and animals were humanely euthanized if tumor burden or health metrics were met as detailed above.

### Statistics

All results are expressed as mean±SEM. Student's t-test was used to compare two groups. Survival rates were analyzed using the log-rank (Mantel-Cox) test. P-values <0.05 were considered as statistically significant. All statistical tests were performed using GraphPad Prism v9.3 (GraphPad Software).

### SUMMARY

Applicant provides herein engineered nanoparticles that have the capability to recruit NK cells. A dual-pronged approach was developed where Applicant successfully combined Cowpea mosaic virus (CPMV), with NK agonists. Applicant also demonstrated that a combination of CPMV and NK cell agonists such as the monoclonal antibody agonist anti-4-1BB is an effective dual therapy approach to improve recruited NK cell function and in situ cancer vaccination efficacy. Using murine models of metastatic colon carcinomatosis and intradermal melanoma, CPMV+anti-4-1BB dual therapy provided a robust anti-tumor response, improved elimination of primary tumors, and reduced mortality compared to CPMV and anti-4-1BB monotherapies. Additionally, there was significant delay/prevention of tumor development and improved survival on tumor rechallenge, highlighting the CPMV and NK cell agonist dual therapy enables potent and durable anti-tumor efficacy.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Goto, T. Radiation as an in situ auto-vaccination: Current perspectives and challenges. Vaccines 7, 100 (2019).
2. Hammerich, L., Bhardwaj, N., Kohrt, H. E. & Brody, J. D. In situ vaccination for the treatment of cancer. Immunotherapy 8, 315-330 (2016).
3. Derynck, R., Turley, S. J. & Akhurst, R. J. TGFβ biology in cancer progression and immunotherapy. Nat. Rev. Clin. Oncol. 18, 9-34 (2021).
4. Au, K. M., Park, S. I. & Wang, A. Z. Trispecific natural killer cell nanoengagers for targeted chemoimmunotherapy. Sci. Adv. 6, eaba8564 (2020).
5. Cao, Y. et al. Immune checkpoint molecules in natural killer cells as potential targets for cancer immunotherapy. Signal Transduct. Target. Ther. 5, 250 (2020).
6. Wang, C., Beiss, V. & Steinmetz, N. F. Cowpea Mosaic Virus Nanoparticles and Empty Virus-Like Particles Show Distinct but Overlapping Immunostimulatory Properties. J. Virol. 93, e00129-19 (2019).
7. Czapar, A. E., Tiu, B. D. B., Veliz, F. A., Pokorski, J. K. & Steinmetz, N. F. Slow-Release Formulation of Cowpea Mosaic Virus for In Situ Vaccine Delivery to Treat Ovarian Cancer. Adv. Sci. (Weinheim, Baden-Wurttemberg, Ger. 5, 1700991 (2018).
8. Lizotte, P. H. et al. In situ vaccination with cowpea mosaic virus nanoparticles suppresses metastatic cancer. Nat. Nanotechnol. 11, 295-303 (2016).

9 Kerstetter-Fogle, A. et al. Plant Virus-Like Particle In Situ Vaccine for Intracranial Glioma Immunotherapy. Cancers (Basel). 11, 515 (2019).
10. Wang, C., Fiering, S. N. & Steinmetz, N. F. Cowpea Mosaic Virus Promotes Anti-Tumor Activity and Immune Memory in a Mouse Ovarian Tumor Model. Adv. Ther. 2, 1900003 (2019).
11. Albakri, M. M., Veliz, F. A., Fiering, S. N., Steinmetz, N. F. & Sieg, S. F. Endosomal toll-like receptors play a key role in activation of primary human monocytes by cowpea mosaic virus. Immunology 159, 183-192 (2020).
12. Hoopes, P. J. et al. Treatment of canine oral melanoma with nanotechnology-based immunotherapy and radiation. Mol. Pharm. 15, 3717 (2018).
13. Alonso-Miguel D., Valdivia G., Guerrera D., Perez-Alenza M. D., Pantelyushin S., Alonso-Diez A., Steinmetz N. F., vom Berg J., Beiss V.*, Fiering S., Suarez-Redondo M., Peña L., A.-P. H. Neoadjuvant in situ vaccination with cowpea mosaic virus as a novel therapy against canine inflammatory mammary cancer. J. Immunother. Cancer 10, e004044 (2022).
14. Paul, S. & Lal, G. The Molecular Mechanism of Natural Killer Cells Function and Its Importance in Cancer Immunotherapy. Front. Immunol. 8, 1124 (2017).
15. Vivier, E., Tomasello, E., Baratin, M., Walzer, T. & Ugolini, S. Functions of natural killer cells. Nat. Immunol. 9, 503-510 (2008).
16. Chiossone, L., Dumas, P.-Y., Vienne, M. & Vivier, E. Natural killer cells and other innate lymphoid cells in cancer. Nat. Rev. Immunol. 18, 671-688 (2018).
17. Halstead, E. S., Mueller, Y. M., Altman, J. D. & Katsikis, P. D. In vivo stimulation of CD137 broadens primary antiviral CD8+ T cell responses. Nat. Immunol. 3, 536-541 (2002).
18. Kohrt, H. E. et al. Stimulation of natural killer cells with a CD137-specific antibody enhances trastuzumab efficacy in xenotransplant models of breast cancer. J. Clin. Invest. 122, 1066-75 (2012).
19. Zapata, J. M. et al. CD137 (4-1BB) Signalosome: Complexity Is a Matter of TRAFs. Front. Immunol. 9, 2618 (2018).
20. Wilcox, R. A. et al. Provision of antigen and CD137 signaling breaks immunological ignorance, promoting regression of poorly immunogenic tumors. J. Clin. Invest. 109, 651-9 (2002).
21. Gauttier, V. et al. Agonistic anti-CD137 antibody treatment leads to antitumor response in mice with liver cancer. Int. J. Cancer 135, 2857-2867 (2014).
22. Vinay, D. S. & Kwon, B. S. Therapeutic potential of anti-CD137 (4-1BB) monoclonal antibodies. Expert Opin. Ther. Targets 20, 361-373 (2016).
23. Guillerey, C. et al. Immunosurveillance and therapy of multiple myeloma are CD226 dependent. J. Clin. Invest. 125, 2077-2089 (2015).
24. Wang, C. & Steinmetz, N. F. A Combination of Cowpea Mosaic Virus and Immune Checkpoint Therapy Synergistically Improves Therapeutic Efficacy in Three Tumor Models. Adv. Funct. Mater. 30, 2002299 (2020).
25. Cai, H., Shukla, S. & Steinmetz, N. F. The Antitumor Efficacy of CpG Oligonucleotides is Improved by Encapsulation in Plant Virus-Like Particles. Adv. Funct. Mater. 30, 1908743 (2020).
26. Murray, A. A., Sheen, M. R., Veliz, F. A., Fiering, S. N. & Steinmetz, N. F. In Situ Vaccination of Tumors Using Plant Viral Nanoparticles. Methods Mol. Biol. 2000, 111-124 (2019).
27. Murray, A. A., Wang, C., Fiering, S. & Steinmetz, N. F. In Situ Vaccination with Cowpea vs Tobacco Mosaic Virus against Melanoma. Mol. Pharm. 15, 3700-3716 (2018).
28. Boone, C. E. et al. Active Microneedle Administration of Plant Virus Nanoparticles for Cancer in situ Vaccination Improves Immunotherapeutic Efficacy. ACS Appl. nano Mater. 3, 8037-8051 (2020).
29. Bommareddy, P. K. & Kaufman, H. L. Unleashing the therapeutic potential of oncolytic viruses. J. Clin. Invest. 128, 1258-1260 (2018).
30. Marotel, M., Hasim, M. S., Hagerman, A. & Ardolino, M. The two-faces of NK cells in oncolytic virotherapy. Cytokine Growth Factor Rev. 56, 59-68 (2020).
31. Wu, Y. et al. Recombinant Newcastle disease virus (NDV/Anh-IL-2) expressing human IL-2 as a potential candidate for suppresses growth of hepatoma therapy. J. Pharmacol. Sci. 132, 24-30 (2016).
32. Zamarin, D., Vigil, A., Kelly, K., García-Sastre, A. & Fong, Y. Genetically engineered Newcastle disease virus for malignant melanoma therapy. Gene Ther. 16, 796-804 (2009).
33. Hock, K. et al. Oncolytic influenza A virus expressing interleukin-15 decreases tumor growth in vivo. Surgery 161, 735-746 (2017).
34. Gautam, A., Beiss, V., Wang, C., Wang, L. & Steinmetz, N. F. Plant Viral Nanoparticle Conjugated with Anti-PD-1 Peptide for Ovarian Cancer Immunotherapy. Int. J. Mol. Sci. 22, 9733 (2021).
35. Aljabali, A. A. A., Shukla, S., Lomonossoff, G. P., Steinmetz, N. F. & Evans, D. J. CPMV-DOX delivers. Mol. Pharm. 10, 3-10 (2013).
36. Patel, R., Czapar, A. E., Fiering, S., Oleinick, N. L. & Steinmetz, N. F. Radiation Therapy Combined with Cowpea Mosaic Virus Nanoparticle in Situ Vaccination Initiates Immune-Mediated Tumor Regression. ACS omega 3, 3702-3707 (2018).
37. Chung, Y. H. et al. Integrating plant molecular farming and materials research for next-generation vaccines. Nat. Rev. Mater. 1-17 (2021). doi: 10.1038/s41578-021-00399-5
38. Hoopes, P. J. et al. Treatment of Canine Oral Melanoma with Nanotechnology-Based Immunotherapy and Radiation. Mol. Pharm. 15, 3717-3722 (2018).

1. A composition or combination comprising a plant virus selected from Cowpea chlorotic mottle virus (CCMV), Cowpea mosaic virus (CPMV), Physalis mottle virus (PhMV), and Sesbania mosaic virus (SeMV), and a natural killer (NK) cell agonist.

2. A composition or combination consisting essentially of a plant virus selected from Cowpea chlorotic mottle virus (CCMV), Cowpea mosaic virus (CPMV), Physalis mottle virus (PhMV), Sesbania mosaic virus (SeMV), and a natural killer (NK) cell agonist.

3. The composition or combination of claim 1 wherein the plant virus is a Cowpea mosaic virus (CPMV).

4. The composition or combination of claim 2 wherein the plant virus is a Cowpea mosaic virus (CPMV).

5. The composition or combination of claim 1, wherein the NK cell agonist comprises an anti-4-1BB antibody or an immunogenic fragment of the antibody.

6. The composition or combination of claim 5, wherein the immunogenic fragment of the antibody is selected from Fab, Fab′, F(ab′)₂, Fv, scFv, dsFv, or Fd fragment.

7. The composition or combination of claim 1, wherein the plant virus and the NK agonist are in the same composition.

8. The composition or combination of claim 1, wherein the plant virus and the NK agonist are in the same or different compositions.

9-10. (canceled)

11. The composition or combination of claim 1 comprising a plurality of the plant virus are the same or different from each other.

12. The composition or combination of claim 1 comprising a plurality of NK cell agonists that may be the same or different from each other.

13. (canceled)

14. A method for inducing an immune response or for treating cancer in a subject in need thereof comprising administering to the subject the composition or combination of claim 1.

15-16. (canceled)

17. The method of claim 14, wherein the cancer is selected from melanoma, breast cancer, prostate cancer, lung cancer, ovarian cancer, skin cancer, bladder cancer, pancreatic cancer, gastric cancer, esophageal cancer, head and neck, colon cancer, brain cancer, glioma, cervical cancer, hepatocellular cancer, or thyroid cancer.

18. (canceled)

19. The method of claim 17, wherein the cancer is metastatic or primary lung cancer, ovarian cancer, colon cancer, melanoma or breast cancer.

20-21. (canceled)

22. The method of claim 14, further comprising the administration of a different cancer therapy or tumor resection.

23. The method of claim 14, wherein the administering comprises intravenous, subcutaneous, intratumoral, or intraperitoneal delivery.

24. (canceled)

25. A kit comprising one or more of the composition or combination of claim 1 and optional instructions for use.


SEQUENCE LISTING (5 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 3 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 4 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 5 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN